(19) United States
(12) Patent Application Publication
(10) Pub. No.: US 2016/0000032 A1
(43) Pub. Date: Jan. 7, 2016

(54) ZOYSIA JAPONICA STEUD GIN GI

(71) Applicant: Zoysia Gingi Limited Liability Company, Gwangju (KR)

(72) Inventor: Dong Hyo Byun, Gwangju (KR)

(21) Appl. No.: 14/521,363

(22) Filed: Oct. 22, 2014

(30) Foreign Application Priority Data
Jul. 3, 2014 (KR) 10-2014-0082833

(51) Int. Cl. A01H 5/12

(52) U.S. Cl. CPC A01H 5/12

(57) ABSTRACT

A turfgrass variety, Gin gi, characterized in that the plant length is 1-3 cm, leaf sheath length is 0.5-1.2 cm, leaf sheath color is RHS green group 134A, leaf blade length is 1-3 cm, leaf blade color is RHS green group 141A, and length of root grown from underground stolon is 30-50 cm, in which the leaf sheath and leaf blade have no trichome, green color is maintained until early December, intense propagation occurs without showing any symptom of diseases even under two irrigations per day during summer months due to high humidity resistance, main propagation is based on vegetative propagation of stolon or underground stolon, and propagation is also possible by seed germination. Due to an excellent mat-forming property, it can be used for an area in which covering or greening is required.

### CROSS-REFERENCE TO RELATED APPLICATIONS AND CLAIM OF PRIORITY

The present application claims the benefit of Korean Patent Application No. 10-2014-0082833, filed Jul. 3, 2014 in the Korean Intellectual Property Office, the entire contents of which are incorporated herein by reference.

### BACKGROUND

1. Technical Field

The present invention relates to a new turfgrass variety, Gin gi. More specifically, it relates to “new turfgrass variety Gin gi” characterized in that the plant length is 1-3 cm, leaf sheath length is 0.5-1.2 cm, leaf sheath color is RHS green group 134A, leaf blade length is 1-3 cm, leaf blade color is RHS green group 141A, and length of root grown from underground stolon is 30-50 cm, in which the leaf sheath and leaf blade have no trichome, green color is maintained until early December, intense propagation occurs without showing any symptom of diseases even under two irrigations per day during summer months due to high humidity resistance, main propagation is based on vegetative propagation of stolon or underground stolon, and propagation is also possible by seed germination.

2. Background Art

Turfgrass is a perennial plant of family Gramineae and it is an herbaceous plant with very good texture. It indicates lawn grass of a garden which is closely cut and covers ground surface. It is widely distributed all over the world, i.e., in a hot temperature zone, in a warm temperature zone, and also in a cold temperature zone. There are presently almost 5000 species of 600 genuses and it is classified into warm season turfgrass and cool season turfgrass based on strength of cold resistance under different climate conditions. The warm season turfgrass is mainly cultivated in a dry or humid region of south with warm temperature while the cool season turfgrass is mainly cultivated in a dry or humid region of north with cold temperature. Representative examples of the warm season turfgrass include Korean turfgrass (Zoysia grass) which is naturally found in Korea, and representative examples of the cold season turfgrass include western turfgrass like Kentucky bluegrass and Bentgrass.

Zoysia grass belonging to the warm season turfgrass is naturally found in a wide region of Far East Asia which includes Korea. It has been reported that there are 5 to 6 species of turfgrass in Korea, i.e., Zoysia japonica, Zoysia matrella, Zoysia tenuifolia, Zoysia sinica, and Zoysia macrostachya. Zoysia japonica is strong and constitutes most of the turfgrass in Korea. It is grown to have leaf width of 4-7 mm and height of 10-20 cm. Thus, Zoysia grass is most suitable in terms of weather in Korea, and it has not only excellent adaptability to an environment but also stronger dry resistance, pressure resistance, cold resistance, heat resistance, salt resistance or the like than other cool season turfgrasses.

Recently, as turfgrass is used more than ever before, development of a new variety satisfying various interests, i.e., a variety allowing fast growing and easy growing method, a variety with long survival period, a seed-type variety, or the like, is strongly waited for. In order to satisfy such needs, a number of new vegetative varieties have been suggested. In U.S., there has been an interest in Zoysia grass since 1905 and researches were made by U.S. Agricultural Department and many universities, and as a result, a vegetative propagation variety like Emerald; 1955, Meyer; 1951, Midwest; 1963, or the like and a seed propagation variety like Zenith, J37, J36, or the like were developed and made available for use. However, in Korea, there are only Anyang-Jung gi, Benest 7, Kunhee, and Senoc, and studies for developing a new variety are scarce.

As such, development of a variety satisfying different interests, i.e., a variety allowing fast growing and easy growing method, a variety with long survival period, a seed-type variety, or the like, is strongly waited for and breeding of a new Zoysia grass variety satisfying those requirements and having characteristics like being most suitable for Korean soil and capable of replacing foreign varieties by overcoming short green period is highly demanded.

Furthermore, recently there is a great interest in an environment due to global warming and yellow dust that are caused by climate change, and thus efforts are being made to have environment-friendly city conditions to cope with a change in peoples' lifestyle, and a requirement for having so-called ‘green city’ increases. As a result, a demand for turfgrass which has an optimum trait to achieve a green city increases.

In Korean Patent Registration No. 1016249, “New turfgrass variety Yong Chon” is described, and in Korean Patent Application Publication No. 2013-0083630, “New turfgrass variety Green Joa” is disclosed. However, new turfgrass variety Zoysia japonica Steud Gin gi of the present invention has never been disclosed.

### DETAILED DESCRIPTION

The present invention is devised in view of the needs described above. Specifically, by cultivating a new variety according to cross-breeding of turfgrasses selected from an area near Gawngju and Chonnam province and selection of good seeds and by comparing morphological and molecular genetic characteristics, turfgrass of which leaf color is strong green (RHS 141A), green period is long, and plant height above ground is only 3 cm or less to form an excellent mat is obtained, and it is named “Gin gi”, a new variety of warm season turfgrass.

In order to solve the problems described above, provided by the present invention is new turfgrass variety Gin gi having morphological characteristics that are described below, in which the leaf sheath and leaf blade have no trichome, green color is maintained until early December, intense propagation occurs without showing any symptom of diseases even under two irrigations per day during summer months due to high humidity resistance, main propagation is based on vegetative propagation of stolon or underground stolon, and propagation is also possible by seed germination:
(a) plant length: 1-3 cm
(b) leaf sheath length: 0.5-1.2 cm
(c) leaf sheath color: RHS (Royal Horticultural Society) green group 134A
(d) leaf blade length: 1-3 cm
(e) leaf blade color: RHS (Royal Horticultural Society) green group 141A, and
(f) length of root grown from underground stolon: 30-50 cm.

The new turfgrass variety Gin gi of the present invention has characteristics that leaf greenness is high and growth is limited to short height. Thus, lawn mowing is unnecessary, and due to an excellent mat-forming property, it can be advantageously used for an area in which covering or greening is required like an area under development, a farm, an arena, and a golf course. It also has an effect of being usefully used in an industry related to turfgrass, in particular, for export of excellent varieties of Korean turfgrass. It can be also effectively used for preventing soil loss as the turfgrass has long and strong roots.

### BRIEF DESCRIPTION OF THE DRAWINGS

FIGS. 1A and 1B include photographic images showing the callus (FIG. 1A) and new shoots (FIG. 1B) that have been induced from new variety Gin gi.

FIG. 2 includes photographic images showing an overview of new variety Gin gi grown in a field (top) and close-up shot of the Gin gi grown in a field (bottom).

FIG. 3 includes photographic images showing growth of new variety Gin gi (top) and Zoysia matrella (bottom) as a control. Above-ground height of Gin gi plant was 3 cm or so, which does not require any mowing, and the plant maintains strong green color. However, Zoysia matrella grows high and exhibits pale green color.

FIG. 4 includes photographic images showing the growth of stolon of new variety Gin gi (top) or Zoysia matrella (bottom) as a control. The length of Zoysia matrella is the same or greater than twice the length of Gin gi.

FIG. 5 includes photographic images showing an overview of new variety Gin gi (top) or Zoysia matrella (bottom) grown in a field for a mat-forming test. Only a part of Gin gi exhibited a phenomenon of slight crawling of leaf edges. However, Zoysia matrella exhibited a phenomenon of crawling of a leaf blade after growth of a leaf blade and a flag leaf.

FIG. 6 includes photographic images showing growth of new variety Gin gi (top) and Zoysia matrella (bottom) as a control after single mowing. Gin gi plant kept strong green color without requiring any mowing while Zoysia matrella exhibited not only pale green color but also very fast leaf growth rate.

FIG. 7 includes photographic images showing growth of new variety Gin gi in a field. Most of Gin gi plants were not observed with inflorescence (top), but few plants expressed a growth cycle during which inflorescence is formed (bottom), and thus it was confirmed that seed propagation is also possible.

FIG. 8 includes photographic images showing growth of new variety Gin gi in a field. The photographic images were taken on Nov. 14, 2013, i.e., late autumn, but the turfgrass still exhibits strong green color.

FIGS. 9A and 9B include photographic images showing (FIG. 9A): the result of electrophoresis of genomic DNA, which has been extracted from Gin gi (1), Zoysia matrella (2), or Zoysia japonica (3) and (FIG. 9B): the graph of DNA analysis by using Nanodrop spectrophotometric analysis.

FIG. 10 illustrates the result of observing genetically different specific bands by RAPD (Random amplified polymorphic DNA), which uses a variety-specific primer (OPB-7, OPB-8, OPB-11) for Gin gi(lanes 1, 4, 7), Zoysia matrella (lanes 2, 5, 8), or Zoysia japonica (lanes 3, 6, 9).

### DETAILED DESCRIPTION

In order to solve the problems described above, provided by the present invention is new turfgrass variety Gin gi having morphological characteristics that are described below, in which the leaf sheath and leaf blade have no trichome, green color is maintained until early December, intense propagation occurs without showing any symptom of diseases even under two irrigations per day during summer months due to high humidity resistance, main propagation is based on vegetative propagation of stolon or underground stolon, and propagation is also possible by seed germination:
(a) plant length: 1-3 cm
(b) leaf sheath length: 0.5-1.2 cm
(c) leaf sheath color: RHS (Royal Horticultural Society) green group 134A
(d) leaf blade length: 1-3 cm
(e) leaf blade color: RHS (Royal Horticultural Society) green group 141A, and
(f) length of root grown from underground stolon: 30-50 cm.

On Jul. 1, 2014, the new turfgrass variety Gin gi was duly deposited with Korean Collection for Type Cultures (KCTC) (having the address of Biological Resource Center (BRC), Korea Research Institute of Bioscience and Biotechnology (KRIBB), 52 Eoeun-dong, Yuseong-gu, Daejeon 305-333, Republic of Korea) under the Access number of KCTC 12616BP. The deposit has been made under the terms of the Budapest Treaty and all restrictions imposed by the depositor on the availability to the public of the biological material will be irrevocably removed upon the granting of a patent.

The new variety Gin gi according to one embodiment of the present invention has been obtained as follows: Zoysia matrella, Jung gi, and Zoysia japonica selected from an area near Gwangju and Chonnam province in Korea were subjected to cross-breeding, i.e., bundles of selected varieties were cultivated according to mixed planting, and crossbred variety was induced so as to allow artificial selection and natural breeding, and after selecting favorable seeds, a variant of Zoysia matrella was found based on a breeding test and then cultivated.

The new variety Gin gi according to one embodiment of the present invention is propagated by vegetative propagation of stolon or underground stolon, and although inflorescence and seeds are not generally formed, few plants expressed a growth cycle during which inflorescence and seeds are formed from spring to late autumn. As such, if culture conditions are appropriate, it is believed that seed propagation is also possible in a temperate climate region in addition to vegetative propagation. Further, it is also possible that asexual reproduction can be obtained by a tissue culture method or the like, which is generally known in the art.

Gin gi maintained green color until early December in the region of Gwangju, indicating a long green period. Further, the stolon kept growing until early November. Further, intense propagation occurred without showing any symptom of diseases even under two irrigations per day during summer months due to high humidity resistance.

Further, according to the new variety Gin gi of the present invention, the above-ground plant height is 3 cm or less, and thus not requires any lawn mowing, greenness of the above-ground plant is distinctly stronger than Zoysia japonica as a control (RHS 141A), no trichomes are observed in leaf sheath and leaf blade, the texture is firm and strong, and the angle between a leaf blade and a flag leaf remains at 90° during growth as a single spore but the angle decreases when the mat-forming progresses according to an increase in density, a phenomenon of slight crawling of leaf edges is shown in part of the claimed variety while leaf blade crawling occurs after growth of leaf blade and flag leaf in Zoysia matrella, the color hue becomes stronger according to season change, and the length of root grown from underground stolon is 30-50 cm.

According to the present invention, the new variety Gin gi is characterized in that, it does not exhibit a specific band at 1.5 kb and 1.8 kb when RAPD is performed by using the primer of SEQ ID NO: 1, it does not exhibit a specific band at 1.3 kb and 2.2 kb when RAPD is performed by using the primer of SEQ ID NO: 2, and it does not exhibit a specific band at 1.0 kb and 2.7 kb when RAPD is performed by using the primer of SEQ ID NO: 3.

The RAPD results according to one embodiment of the present invention may be used as evidence which indicate a clear genetic difference of Gin gi of the present invention compared to other turfgrass.

The aforementioned RAPD (Random Amplified Polymorphic DNA) is regarded as a PCR-based marker, and it is a maker for amplifying and analyzing by PCR a portion of genomic DNA which corresponds to a primer of about 10-mer. Unlike RFLP (Restriction Fragment Length Polymorphism), it allows a sufficient amount of amplified DNA, and thus DNA mutation can be analyzed by a treatment of isolated gel with ethidium bromide, i.e., without requiring an radioisotope or a chemically-labelled probe and a hybridization step. Herein below, the present invention is explained in view of the examples. However, the following examples are given only to illustrate the present invention and by no means the scope of the present invention is limited to them.

### EXAMPLE 1

### Collection and Cultivation of New Turfgrass Variety Gin gi

As it is described in the following Table 1, turfgrass with strong green color was selected from an area near Gawngju and Chonnam province in Korea for cultivation of a new turfgrass variety. Varieties showing strong leaf color were selected, specifically, 5 kinds of Zoysia japonica and two kinds of Zoysia matrella were selected and a bundle of the same type was subjected to a mixed cultivation from July 2009 to May 2012 and induced to yield a cross-breed according to open pollination. That is, during every early October starting from 2009, 5 kinds of Zoysia japonica and 2 kinds of Zoysia matrella were washed, bundled with a twine, and planted in a field. The bundle grown in the field was then again transplanted in a styrofoam box and cultivated until the end of April in a greenhouse to abolish the dormant period. By doing so, the limitation of not being able to observe inflorescence and seeds from Zoysia matrella can be overcome. As a result of allowing natural cross-breeding by having an artificial environment for open pollination, around the same time in March an occurrence of inflorescence and seed formation were observed from Zoysia japonica and Zoysia matrella grown in a greenhouse. Among them, a natural crossbreed with short height was selected and subjected to inbreeding starting from May 2012, and as a result, it was able to confirm that characteristics of the natural crossbreed are maintained at constant level. The turfgrass was propagated by vegetative propagation in a field located in Gwangju (Soe-gu, Chipyong-dong).

TABLE 1

| | |
|---|---|
| Collection area | Chonlanam-do Changsong-gun Buki-myon |
| | Chonlanam-do Changsong-gun Bukha-myon |
| | Chonlanam-do Changsong-gun Samso-myon |
| | Chonlanam-do Changsong-gun Samge-myon |
| | Chonlanam-do Kochang-gun Kochang-up |
| | Gwangju, Chipyong-dong |
| Collection criteria | Turfgrass having leaf color stronger than RHS 134 |
| Collected turfgrass | 7 types (5 types of Zoysia japonica and 2 types of |
| variety | Zoysia matrella) |

During the cultivation period of from 2012 to 2013, the plants were never exposed to disease or insects, and they have stability as only the single form has grown and propagated with no expression of an isoform. Further, as determined from data of molecular biological analysis of mutation in new turfgrass variety Gin gi based on RAPD (Random Amplified Polymorphic DNA), they have distinct properties that are clearly distinguished from those of Zoysia japonica and Zoysia matrella. Further, homogeneity in appearance (i.e., density, color, and mowing height) is maintained. Further, no physiological problem has occurred in the field irrigated twice per day or the field with no irrigation. However, the growth rate was at least twice in the field with irrigation. Due to strong humidity resistance, the propagation was intense without having any symptom of diseases in spite of two irrigational operations per day during summer months. To increase the growth rate, artificial irrigation is frequently required from the end of April. However, during the winter months (i.e., December to end of March), it was confirmed that one irrigation per week is sufficient.

For examination of the characteristics of the variety and development of a DNA marker, the obtained seeds, i.e., Gin gi, were subjected to tissue culture (plant cell culture and callus) for performing a study on characteristics exhibition in an individual plant. Thereafter, deposition was made with Korea Research Institute of Bioscience and Biotechnology on July 1s^{t}, 2014 (Accession number: KCTC 12616BP).

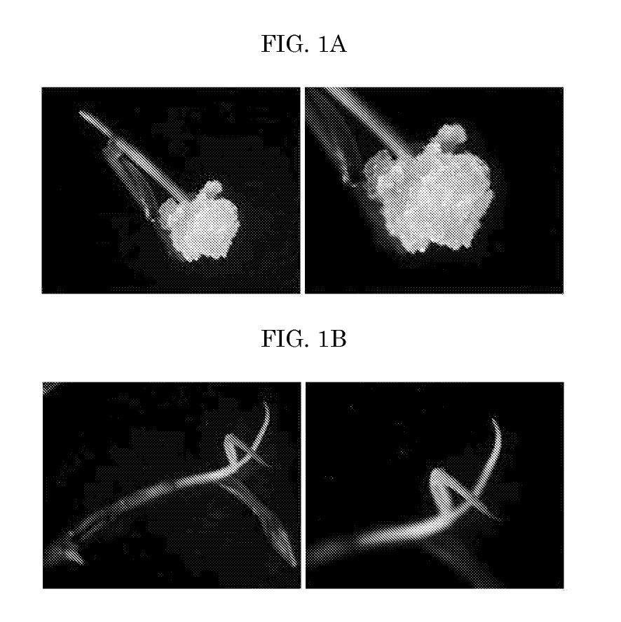

Callus induction from turfgrass was performed according to the following method. The obtained turfgrass sample was fully washed using running water and cut around joint regions by scissors. The turfgrass sample after cutting was subjected to surface sterilization by shaking it for 20 minutes in 10% solution of commercially available bleach. The turfgrass sample obtained after surface sterilization was washed three times with sterilized water. Moisture remained on the turfgrass sample was removed on a sterile bench by using a sterile filter paper. The sample was then cut to a size of about 5 mm by using a mess and planted on a medium for callus induction. As for the medium for callus induction, MS (Murashige & Skoog) medium added with 1 mg/1 of 2,4-D was used as a basic medium. Fragment of the turfgrass joint was placed on each medium and the presence or absence of callus formation was observed under a microscope at an interval of about 1 week. In addition, for inducing new shoots from the joint fragment of the turfgrass, the joint fragment was cultured in MS (Murashige & Skoog) medium added with 1 mg/ml of BA. Each culture corresponds to dark culture in an incubator at 25-26° C. After culturing for 4 weeks approximately, it was found that white cell clusters are formed from the turfgrass joint fragment which has been cultured in a medium added with 2,4-D. After that, according to subculture using the same medium, calluses were propagated (FIG. 1A).

### EXAMPLE 2

### Analysis of Morphological Change in New Turfgrass Variety Gin gi

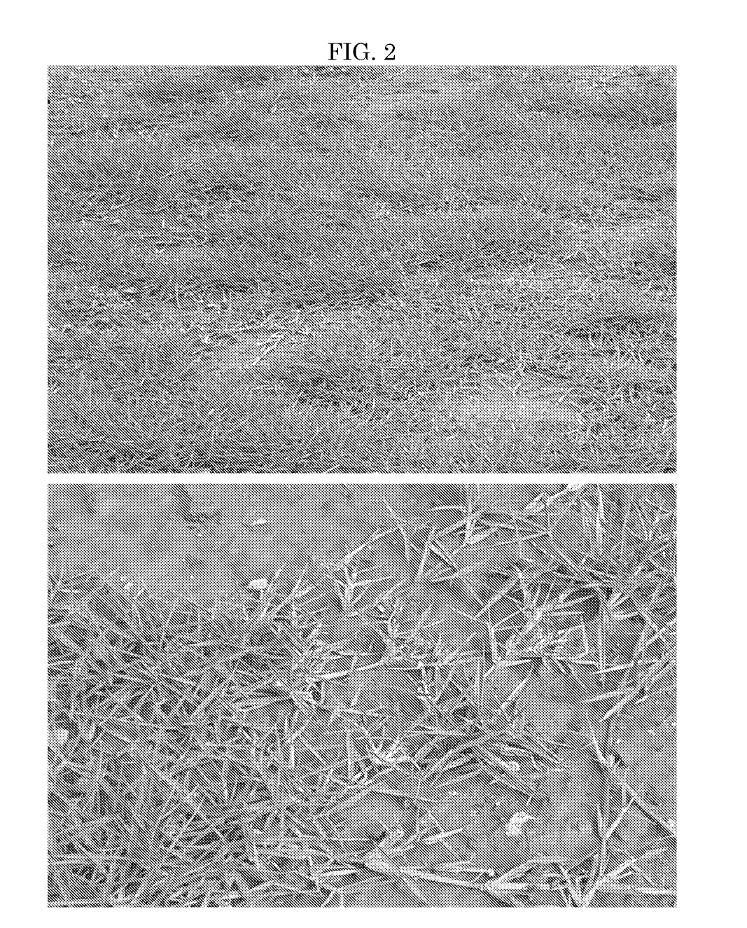

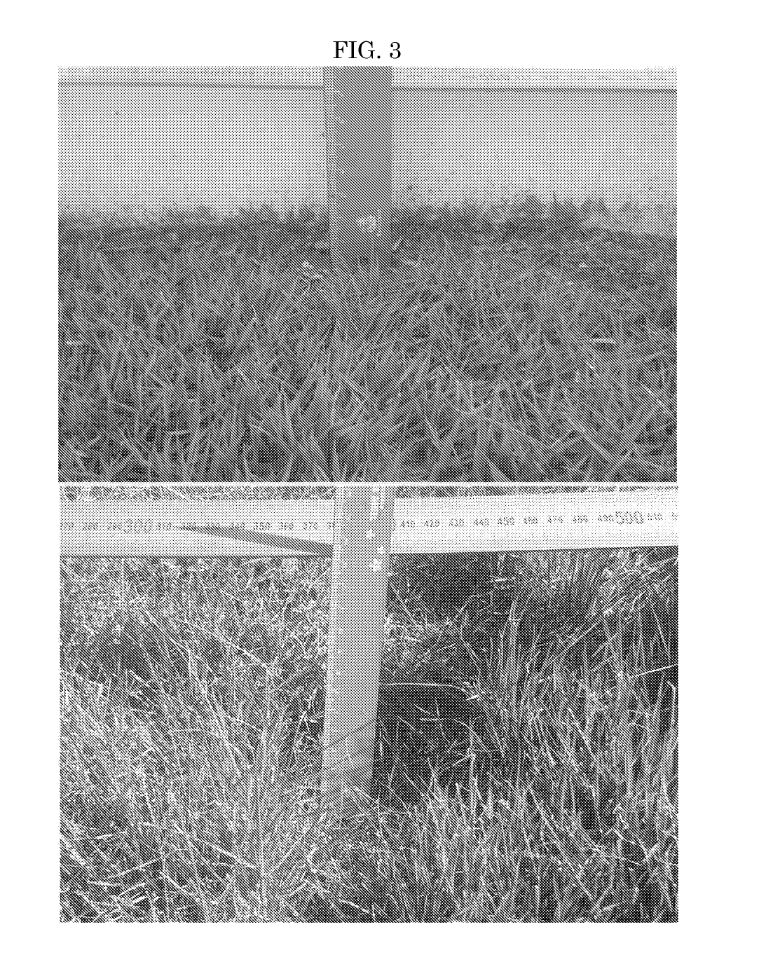

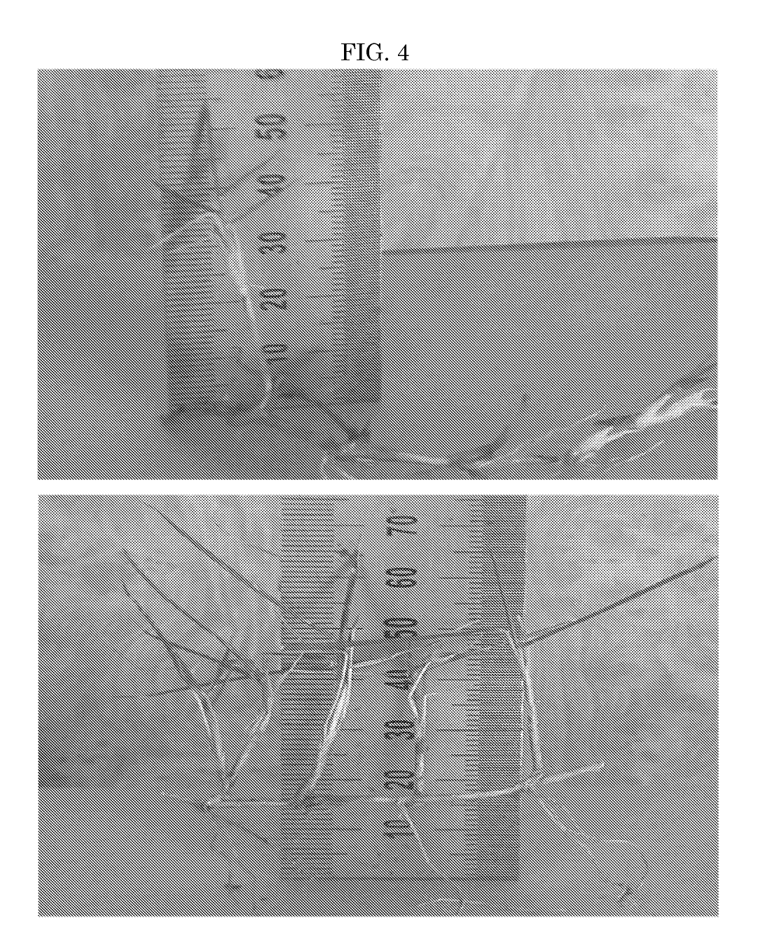

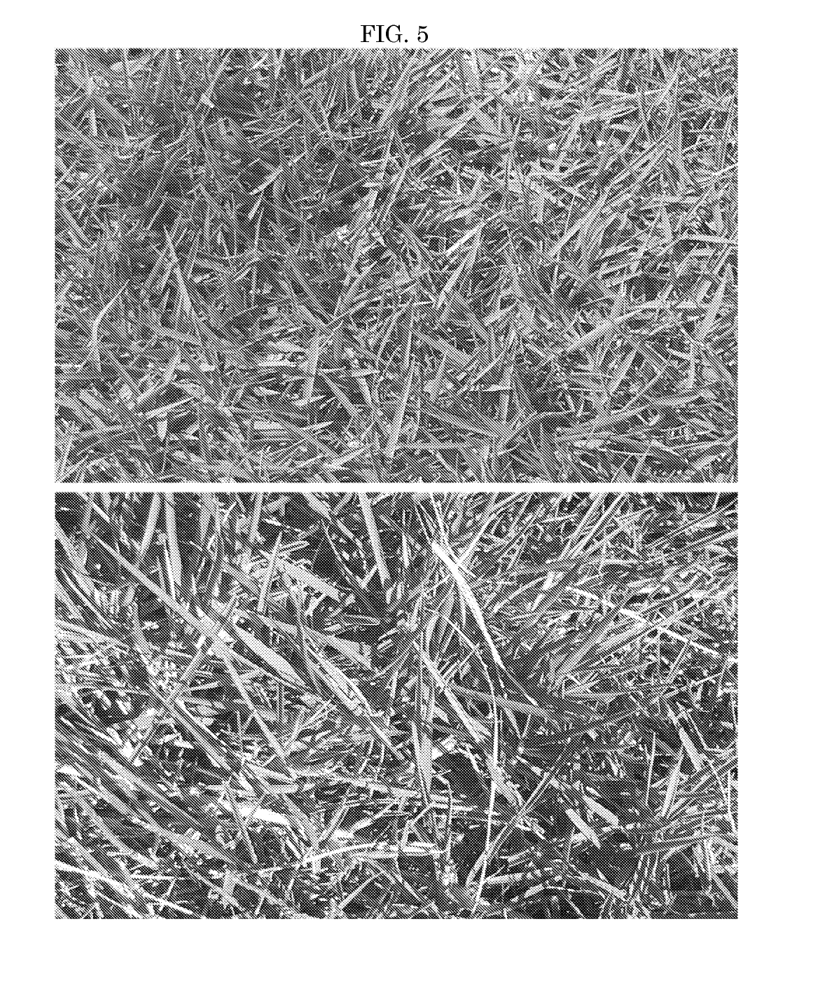

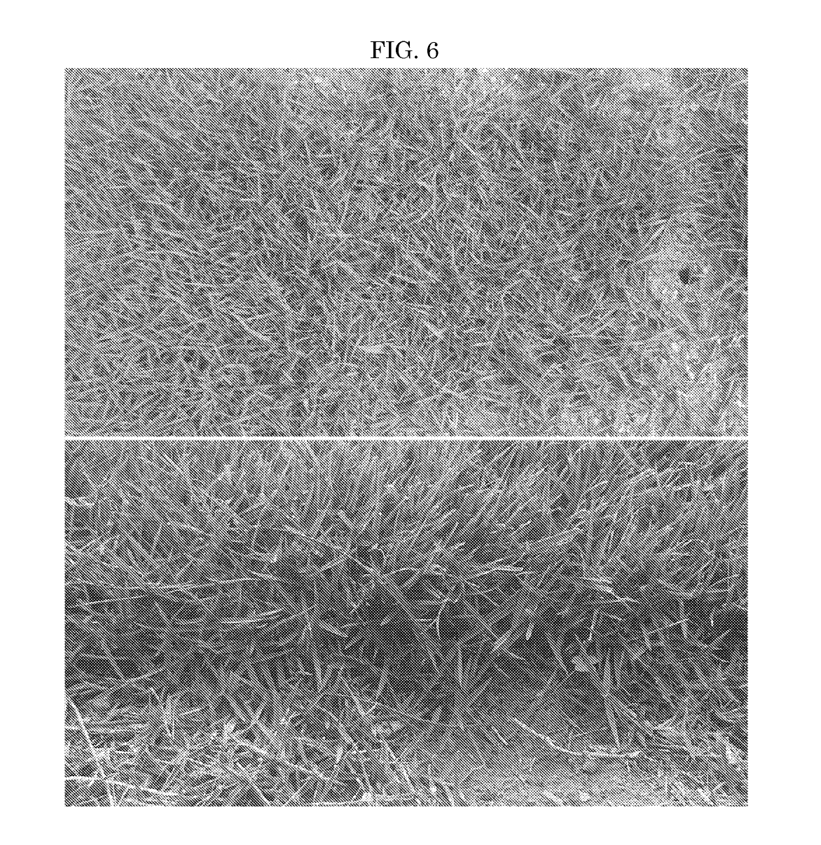

Morphological characteristics of the new variety Gin gi, which is a variant of Korean Zoysia japonica selected from Example 1 above, and Zoysia matrella as a control were tested and observed in the field. The results are summarized in the following Table 2 and FIGS. 2 to 8.

TABLE 2
Comparison of morphological characteristics between new variety Gin gi and control variety

| | | Control variety | |
|---|---|---|---|
| Characteristics | Gin gi | (Zoysia matrella) | Remarks |
| Plant height | 3 cm or less | 5-7 cm | Short |
| Stem shape of young plant | 0.5-1 cm | 1-3 cm | Thin |
| Length of leaf sheath | 1.2 cm or less | 1.5 cm or less | |
| Color of leaf sheath | Green group 134A | Green group 134B | Measured by using Rhs chart |
| | | | of Chonnam Agricultural |
| | | | Technical Center |
| Trichome density on base of | Impossible to observe | Slightly scarce | |
| blade sheath | | | |
| Length of leaf blade | 3 cm or less | 3-10 cm | |
| Width of leaf blade | 4 mm or less | 4 mm or less | |
| Greenness level of leaf blade | Green group 141A | Green group 134A | |
| Trichome density on leaf blade | Scarce | Slightly scarce | |
| surface | | | |
| Trichome density on back | Impossible to observe | Slightly scarce | |
| surface of leaf blade | | | |
| Angle between leaf blade and | Narrow | Medium | Refer to photographic image |
| flag leaf | | | |
| Length of flag leaf | 3 cm or less | 3-10 cm | |
| Width of flag leaf | 4 mm or less | 4 mm or less | |
| Length of inflorescence | 2 cm or less | | |
| Width of inflorescence | 2 mm or less | | |
| Upright angle between flower | Upright, 90° | | |
| stalk and ground surface | | | |
| Ear length | 2 cm or less | | |
| Number of spikelet | 10-16 | | |
| Anthocyanin coloration level in | Rhs 152c | | |
| mature spikelet | | | |
| Anthocyanin coloration level in | Rhs 150d | | |
| immature spikelet | | | |
| Number of hermaphrodite flower | 10-16 | | |
| Number of male flower | 10-16 | | |
| Number of female flower | 10-16 | | |
| Anther length | 5 mm or less | | |
| Anther width | 5 mm or less | | |
| Stamen stem length | 5 mm or less | | |
| Pistil length | 3 mm or less | | |
| Ovary length | 1 mm or less | | |
| Shape of fruit | Linear | | |

Because it was impossible to have natural observation of inflorescence and seeds from the control variety, some data are missing for the control variety.

Although at early germination stage the above-ground plant of the new variety Gin gi of the present invention is similar to Zoysia matrella as a control, the mature plant exhibited characteristic appearances of Zoysia japonica and Jung gi. The most closely related variety in terms of the appearance is Zoysia matrella, and comparisons were subsequently made with Zoysia matrella, Zoysia japonica, and Jung gi.

As a result of the investigation, it was found that Gin gi has vegetative propagation using stolon and underground stolon, and as the growth is limited to the above-ground height of 3 cm or less, no mowing is required. Further, it was also confirmed that the greenness of the above-ground plant is constantly strong (RHS 141A) without any change, which is clearly distinguished from that of Zoysia matrella as a control variety (FIGS. 2 to 8).

In case of Gin gi, characteristics as follows were confirmed (Tables 2 to 4): no trichome is observed from leaf sheath and leaf blade, the texture is good and strong, and the length of root grown from underground stolon is 30-50 cm.

As shown in FIG. 5, when the leaf blade of Gin gi is grown as a single spore, it maintains an angle of 90° relative to a flag leaf but the angle decreases when mat-forming progresses according to an increase in density. Meanwhile, in case of Zoysia matrella as a control, leaf blade crawling occurs after growth of a leaf blade and a flag leaf, but a phenomenon of slight crawling of leaf edges is shown in part of the new variety.

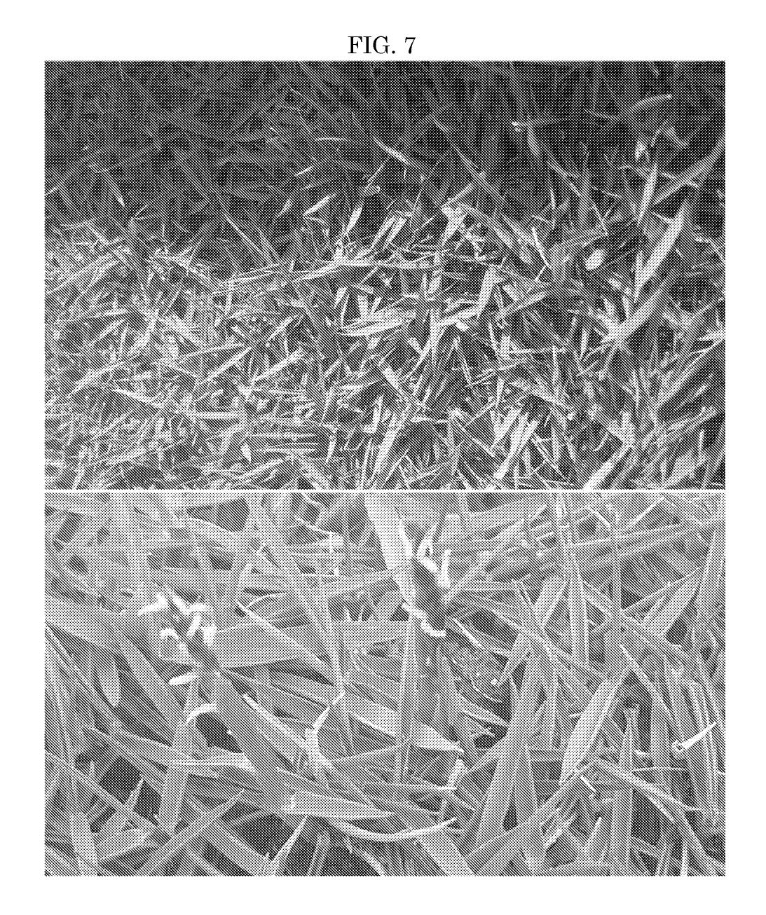

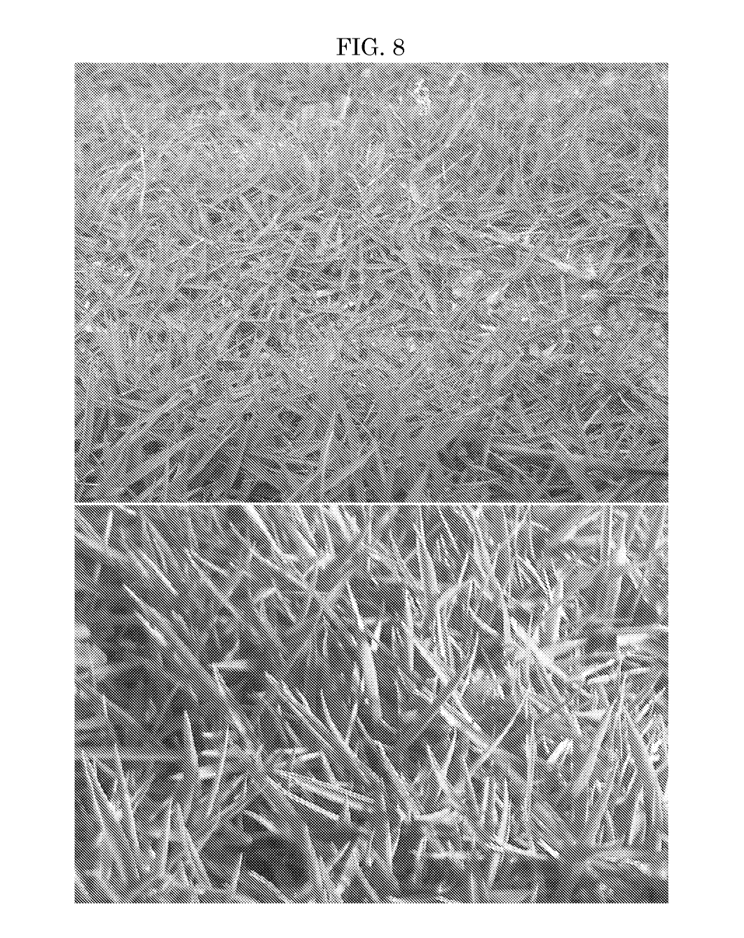

Further, although generally there is no inflorescence and seeds obtained from Gin gi, a few individual plants exhibited a growth period in which inflorescence and seeds are yielded from spring to late autumn, and thus it was confirmed that even in a temperate climate region the seed propagation is possible in addition to vegetative propagation, as long as culture conditions are satisfied (FIG. 7). Gin gi exhibited a long period with strong green color, had stolon which grows until early November, and the color hue became stronger according to a change of season (FIG. 8).

### EXAMPLE 3

### Extraction of Genomic DNA from New Turfgrass Variety Gin gi

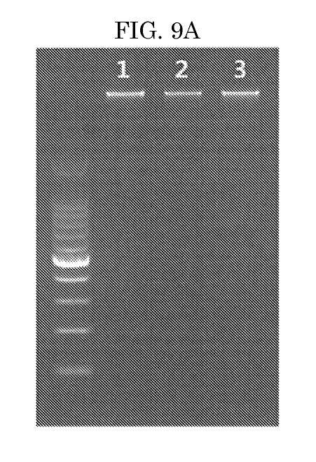

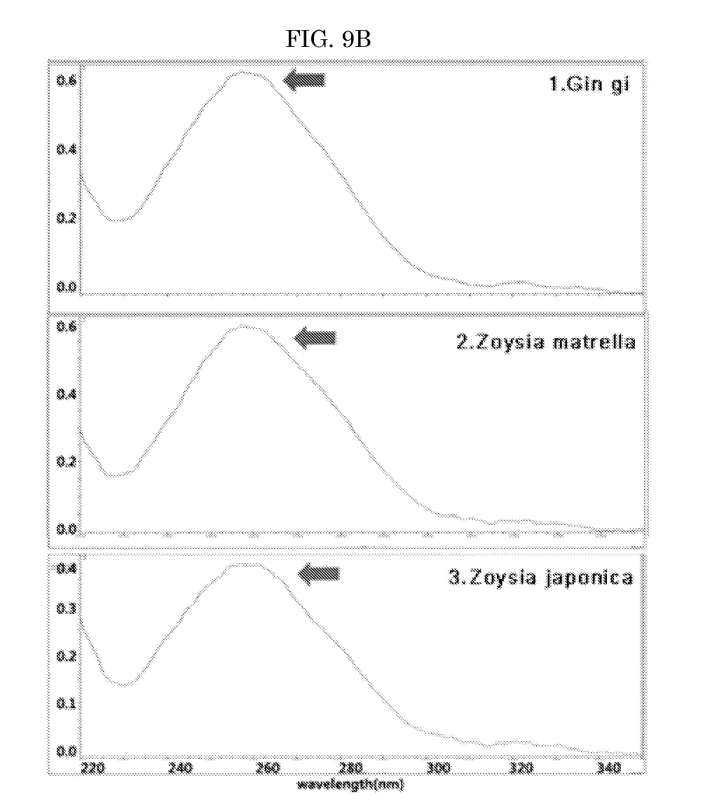

Leaves were collected from each of Gin gi, Zoysia japonica, and Zoysia matrella and crushed in a pestle and mortar by mixing with liquid nitrogen. The crushed leaves were transferred to a 1.5 ml tube and DNA was extracted therefrom according to the protocol of DNeasy Plant Mini kit (Qiagen). Briefly described, 400 μl of AP1 buffer and 4 μl RNase A solution (100 mg/ml) were added to the crushed leaves and admixed with each other followed by keeping for 10 minutes in an incubator at 65° C. 130 μl of P3 buffer was added and culture was performed on ice for 5 minutes. After that, it was centrifuged at 14000 rpm for 5 minutes. The reaction product was added to QlAshredder Mini spin column and then centrifuged for 2 minutes at 14000 rpm. 450 μl of the eluted solution was transferred to a 1.5 ml tube and added and admixed with 675 μl of AW1 buffer. 650 μl of the mixture was added to the spin column and centrifuged for 1 minute at 8000 rpm. The eluted solution was discarded. For washing the DNA separated by spin column, 500 μl of AW2 buffer was added and centrifuged for 1 minute at 8000 rpm. It was again centrifuged for 2 minutes at 14000 rpm to dry the spin column membrane. The spin column was transferred to a 1.5 ml tube, added with 100 μl of AE buffer, and kept at room temperature for 5 minutes. It was then centrifuged at 8000 rpm to extract genomic DNA. The extracted genomic DNA was loaded onto a 1.5% (w/v) agarose gel to confirm the presence of the DNA (FIG. 9A). Then, the result was analyzed by Nanodrop spectrophotometric analysis as shown in Table 3 and FIG. 9B. As a result, the presence of the genomic DNA peak was confirmed, and it indicates that the DNA has been extracted without any problem.

TABLE 3

| | | DNA | | | Extracted |
|---|---|---|---|---|---|
| Serial | | Concentration | | | Amount |
| No. | Sample Name | (ng/μl) | 260/280 | 260/230 | (ng) |
| 1 | Gin gi | 30.1 | 1.86 | 3.07 | 1505 |
| 2 | Zoysia matrella | 29.4 | 1.74 | 3.61 | 1470 |
| 3 | Zoysia japonica | 18.9 | 1.76 | 2.78 | 945 |

### EXAMPLE 4

### Molecular Biological Analysis of Mutation in New Turfgrass Variety Gin gi Based on RAPD (Random Amplified Polymorphic DNA)

Molecular genetic characteristics of Gin gi were determined by RAPD method using a variety-specific primer in which Zoysia japonica and Zoysia matrella as a control and the new variety Gin gi of the present invention are tested. The genomic DNA extracted from above example was used as a PCR template for RAPD analysis. According to PCR which uses Minicycler (M J Research Inc.), the extracted DNA was amplified. As for the PCR reaction solution, 10 ng of DNA, 2 μM of primer, 2> Multiplex PCR Master Mix buffer, and Q-Solution were admixed with another and the reaction volume was adjusted to 25 μl. PCR reaction conditions are as follows: 95° C. (5 minutes), [95° C. (1 minute), 38° C. (1 minute and 30 seconds), 72° C. (2 minutes), 45 cycles], and 68° C. (10 minutes). After admixing 5 μl of the PCR reaction product with 1 μl of Gelstar nucleic acid dye solution, it was electrophoresed at 50 V for 3 hours on a 1.5% (w/v) agarose gel containing 1×TAE. After that, the amplified DNA was photographed using Gel document system (Vilber).

For the PCR described above, OPB-07 (5′-GGTGACGCAG-3; SEQ ID NO: 1), OPB-08 (5′-GTCCACACGG-3; SEQ ID NO: 2) and OPB-11 (5′-GTAGACCCGT-3; SEQ ID NO: 3) primers (Bioneer) were used.

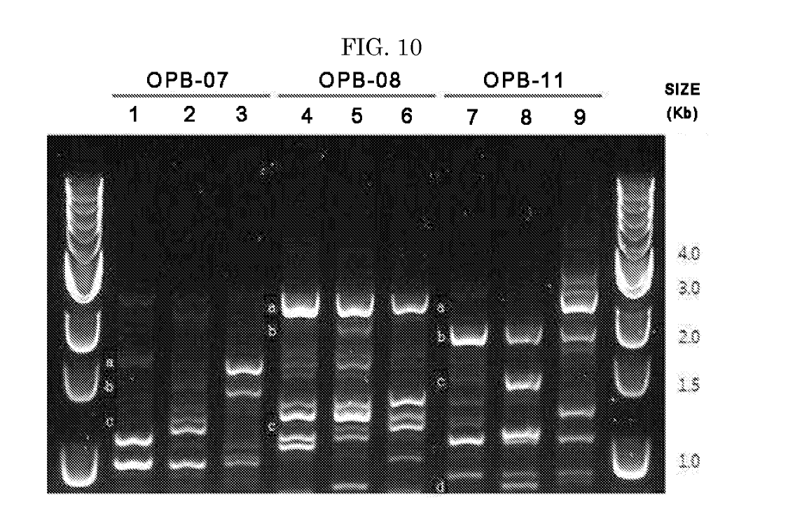

The RAPD PCR result of the new turfgrass variety Gin gi is summarized in the following Table 4 and FIG. 10. According to the result of analysis which uses OPB-07, Gin gi was not observed with “b, c”, unlike Zoysia japonica and Zoysia matrella. According to the result of analysis which uses OPB-08, Gin gi was not observed with “b, c”, unlike Zoysia japonica and Zoysia matrella. According to the result of analysis which uses OPB-011, Gin gi was not observed with “a, d”, unlike Zoysia japonica and Zoysia matrella. Taken together, as Gin gi was observed to have a genomic DNA type which is different from that of Zoysia japonica and Zoysia matrella when the analysis is made by using OPB-07, OPB-08, and OPB-11, it was clearly found to be a new variety.

TABLE 4
Polymorphism band data obtained by RAPD
analysis of new variety Gin gi

| | OPB-7 | | | OPB-8 | | | OPB-11 | | | |
|---|---|---|---|---|---|---|---|---|---|---|
| Turfgrass type | a | b | c | a | b | c | a | b | c | d |
| Gin gi | 1 | 0 | 0 | 1 | 0 | 0 | 0 | 1 | 1 | 0 |
| Zoysia matrella | 1 | 1 | 1 | 1 | 1 | 1 | 0 | 1 | 1 | 1 |
| Zoysia japonica | 1 | 1 | 0 | 1 | 0 | 1 | 1 | 1 | 0 | 0 |

1 indicates presence of the band, and 0 represents absence of the band.

What is claimed is:

1. A turfgrass variety Gin gi having morphological characteristics (a) to (f) that are described below, in which the leaf sheath and leaf blade have no trichome, green color is maintained until early December, intense propagation occurs without showing any symptom of diseases even under two irrigations per day during summer months due to high humidity resistance, main propagation is based on vegetative propagation of stolon or underground stolon, and propagation is also possible by seed germination:
(a) plant length: 1-3 cm
(b) leaf sheath length: 0.5-1.2 cm
(c) leaf sheath color: RHS (Royal Horticultural Society) green group 134A
(d) leaf blade length: 1-3 cm
(e) leaf blade color: RHS (Royal Horticultural Society) green group 141A, and
(f) length of root grown from underground stolon: 30-50 cm.

2. The turfgrass variety Gin gi according to claim 1, characterized in that the new turfgrass variety Gin gi is deposited under Accession number: KCTC 12616BP.

3. The turfgrass variety Gin gi according to claim 1, characterized in that the new turfgrass variety Gin gi does not exhibit a specific band at 1.5 kb and 1.8 kb when RAPD (Random Amplified Polymorphic DNA) is performed by using the primer of SEQ ID NO: 1, does not exhibit a specific band at 1.3 kb and 2.2 kb when RAPD is performed by using the primer of SEQ ID NO: 2, and does not exhibit a specific band at 1.0 kb and 2.7 kb when RAPD is performed by using the primer of SEQ ID NO: 3.


SEQUENCE LISTING (3 sequences)

SEQ ID NO: 1 - DNA, 10 bp, Artificial Sequence
  OPB-07 primer
ggtgacgcag

SEQ ID NO: 2 - DNA, 10 bp, Artificial Sequence
  OPB-08 primer
gtccacacgg

SEQ ID NO: 3 - DNA, 10 bp, Artificial Sequence
  OPB-11 primer
gtagacccgt